(19) United States
(12) Patent Application Publication
(10) Pub. No.: US 2023/0057569 A1
(43) Pub. Date: Feb. 23, 2023

(54) PIVC-INTEGRATED HEMOLYSIS-REDUCTION ACCESSORIES WITH ANTI-SPILLAGE COMPONENT FOR DIRECT BLOOD DRAW

(71) Applicant: CareFusion 303, Inc., San Diego, CA (US)

(72) Inventors: Amarsinh Deeliprao JADHAV, Bangalore (IN); Abin AUSTIN, Thrissur (IN)

(21) Appl. No.: 17/890,977

(22) Filed: Aug. 18, 2022

Related U.S. Application Data
(60) Provisional application No. 63/235,122, filed on Aug. 19, 2021.

(51) Int. Cl. A61M 25/00; A61M 25/06

(52) U.S. Cl. CPC A61M 25/0097; A61M 25/0606; A61M 25/0693; A61M 25/0014

(57) ABSTRACT

A flow restriction device may include a proximal housing including an internal fluid channel, a distal housing including an internal fluid channel, and an intermediate housing interposed between the proximal and distal housings. The intermediate housing may include an internal chamber, and a slider reciprocally disposed in the internal chamber. The slider may include an internal fluid channel and a seal overlaying an outer surface of slider excluding the fluid channel, and may be reciprocally movable between (i) a first position where the internal fluid channel of the slider is axially aligned with the internal fluid channels of the proximal and distal housings to allow a fluid to flow therethrough, and (ii) a second position where the internal fluid channel of the slider is not aligned with the internal fluid channels of the proximal and distal housings and the seal blocks fluid connection between the proximal and distal housings.

### CROSS-REFERENCE TO RELATED APPLICATIONS

This application claims the benefit of U.S. Provisional Application No. 63/235,122, titled “PIVC-INTEGRATED HEMOLYSIS-REDUCTION ACCESSORIES WITH ANTI-SPILLAGE COMPONENT FOR DIRECT BLOOD DRAW,” filed Aug. 19, 2021, the disclosure of which is incorporated herein by reference in its entirety.

### TECHNICAL FIELD

The present disclosure generally relates to blood draw and administration of parenteral fluids to a patient, and particularly to systems and methods to reduce hemolysis in PIVC blood draw whilst reducing blood spillage.

### BACKGROUND

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of catheter is an over-the-needle peripheral intravenous (“IV”) catheter (PIVC). As its name implies, the over-the-needle catheter may be mounted over an introducer needle having a sharp distal tip. A catheter assembly may include a catheter hub, the catheter extending distally from the catheter hub, and the introducer needle extending through the catheter. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal or fluid infusion.

For blood withdrawal or collecting a blood sample from a patient, a blood collection container may be used. The blood collection container may include a syringe. Alternatively, the blood collection container may include a test tube with a rubber stopper at one end. In some instances, the test tube has had all or a portion of air removed from the test tube so pressure within the test tube is lower than ambient pressure. Such a blood collection container is often referred to as an internal vacuum or a vacuum tube. A commonly used blood collection container is a VACUTAINER® blood collection tube, available from Becton Dickinson & Company.

The blood collection container may be coupled to the catheter. When the blood collection container is coupled to the catheter, a pressure in the vein is higher than a pressure in the blood collection container, which pushes blood into the blood collection container, thus filling the blood collection container with blood. A vacuum within the blood collection container decreases as the blood collection container fills, until the pressure in the blood collection container equalizes with the pressure in the vein, and the flow of blood stops.

Unfortunately, as blood is drawn into the blood collection container, red blood cells are in a high shear stress state and susceptible to hemolysis due to a high initial pressure differential between the vein and the blood collection container. Hemolysis may result in rejection and discard of a blood sample. The high initial pressure differential can also result in catheter tip collapse, vein collapse, or other complications that prevent or restrict blood from filling the blood collection container. Furthermore, blood spillage during and/or after blood draw commonly occurs.

The description provided in the background section should not be assumed to be prior art merely because it is mentioned in or associated with the background section. The background section may include information that describes one or more aspects of the subject technology.

### SUMMARY

Aspects of the present disclosure provide a flow restriction device, comprising: a proximal housing configured to couple to a fluid collection device and including an internal fluid channel extending transversely at least partially therethrough; a distal housing configured to couple to a catheter assembly and including an internal fluid channel extending transversely at least partially therethrough; and an intermediate housing interposed between the proximal and distal housings, the intermediate housing comprising: an internal chamber; and a slider reciprocally disposed in the internal chamber, the slider comprising an internal fluid channel extending transversely therethrough and a seal overlaying an outer surface of slider excluding the internal fluid channel, wherein the slider is reciprocally movable between (i) a first position where the internal fluid channel of the slider is axially aligned with the internal fluid channels of the proximal and distal housings to allow a fluid to flow from the distal housing to the proximal housing via the intermediate housing, and (ii) a second position where the internal fluid channel of the slider is not aligned with the internal fluid channels of the proximal and distal housings and the seal blocks fluid connection between the proximal and distal housings.

In some instances, the present disclosure provides methods of manufacturing a flow restriction device, comprising: molding a proximal housing with an internal fluid channel extending transversely at least partially therethrough; molding a distal housing with an internal fluid channel extending transversely at least partially therethrough; and molding an intermediate housing with an internal chamber extending through an outer surface of the intermediate housing; molding a slider with an internal fluid channel extending transversely therethrough; overmolding a seal over at least a portion of the slider excluding the internal fluid channel; mounting the slider with overmolded seal in the intermediate housing; and coupling the intermediate housing between the proximal and distal housings.

In some instances, the present disclosure provides a blood collection system, comprising: a blood collection device; a catheter assembly; and a flow restriction device fluidly coupled to the blood collection device, the flow restriction device comprising: an intermediate housing comprising: an internal chamber and an internal fluid channel extending therethrough on opposing sides of the internal chamber; and a slider reciprocally mounted in the internal chamber, the slider comprising an internal fluid channel extending transversely therethrough and a seal overlaying an outer surface of slider excluding the internal fluid channel; a distal housing coupled to the catheter assembly and including an internal fluid channel extending at least partially therethrough; and a proximal housing coupled to the blood collection device and including an internal fluid channel extending at least partially therethrough, wherein, in a non-actuated state of the slider the internal fluid channel of the slider is not aligned with the internal fluid channels of the proximal, distal, and intermediate housings and the seal blocks fluid connection between the proximal and distal housings.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

The following figures are included to illustrate certain aspects of the embodiments and should not be viewed as exclusive embodiments. The subject matter disclosed is capable of considerable modifications, alterations, combinations, and equivalents in form and function, as will occur to those skilled in the art and having the benefit of this disclosure.

FIG. 1 illustrates a vascular access device including a peripheral intravenous catheter (PIVC) assembly that includes a flow restriction device, in accordance with some embodiments of the present disclosure.

FIG. 2A illustrates a perspective view of the flow restriction device, in accordance with some embodiments of the present disclosure.

FIG. 2B illustrates a cross-sectional view of the flow restriction device of FIG. 2A, in accordance with some embodiments of the present disclosure.

FIG. 3A illustrates a perspective view of a proximal housing of the flow restriction device, in accordance with some embodiments of the present disclosure.

FIG. 3B illustrates a cross-sectional view of the proximal housing of FIG. 3A, in accordance with some embodiments of the present disclosure.

FIG. 4A illustrates a perspective view of a distal housing of the flow restriction device, in accordance with some embodiments of the present disclosure.

FIG. 4B illustrates a cross-sectional view of the distal housing of FIG. 4A, in accordance with some embodiments of the present disclosure.

FIG. 5A illustrates a perspective view of an intermediate housing of the flow restriction device, in accordance with some embodiments of the present disclosure.

FIG. 5B illustrates a cross-sectional view of the intermediate housing of FIG. 5A, in accordance with some embodiments of the present disclosure.

FIG. 6A illustrates a perspective view of a slider of the flow restriction device, in accordance with some embodiments of the present disclosure.

FIG. 6B illustrates a cross-sectional view of the slider of FIG. 6A, in accordance with some embodiments of the present disclosure.

FIG. 7 illustrates a cross-sectional view of the flow restriction device in a non-actuated state of the slider, in accordance with some embodiments of the present disclosure.

FIG. 8 illustrates a cross-sectional view of the flow restriction device in an actuated state of the slider, in accordance with some embodiments of the present disclosure.

FIG. 9 illustrates a cross-sectional view of the flow restriction device returned to the non-actuated state of the slider after completion of fluid draw, in accordance with some embodiments of the present disclosure.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions may be provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

Blood draw via a vascular access device has drawn increasing attention attributed to minimized needle sticks and improved operation efficiency as compared with traditional blood draw methods with venipuncture. Current blood draw using a peripheral intravenous catheter (PIVC) has seen some challenges, one of the most critical is hemolysis related blood quality. In particular, with currently existing PIVC products in the market, along with the standard connection (such as a short extension set and a needleless connector), and blood collection devices (such as a Vacutainer), the shear stress exerted onto blood cells tends to be on the verge of hemolyzing.

Various embodiments of the present disclosure are directed to providing systems and methods to address hemolysis in PIVC blood draw with a hemolysis reduction accessory (also referred to herein as a flow restriction device) which is pre-attached to the PIVC and serves as a flow restrictor to reduce risk of hemolysis. The hemolysis-reduction accessory is advantageously compatible with PIVC placement and does not necessitate change to any of the existing operations. The hemolysis-reduction accessory of the various embodiments described herein is potentially applicable to a wide variety of PIVC products, and compatible with existing blood collection devices and infusion disposables.

Various embodiments of the present disclosure focus on effective flow restriction with the add-on hemolysis-reduction accessory (also referred to herein as a flow restriction device) that regulates the overall flow rate of the entire fluid path as blood cells travel through. The flow restriction device can be either assembled with the PIVC or co-packaged with the PIVC. As such, there is no additional operation during catheter placement since the device has a vented lumen that enables blood flashback. The clinician may connect a blood collection device to the port of the accessory and can then draw blood to the intended volume. After blood draw, the clinician may disconnect and discard the flow restriction device and the blood collection device together. As such, this flow restriction device can be either for single blood draw or stay inline throughout indwell.

In some embodiments, the flow restriction device may include a proximal housing, a distal housing, an intermediate housing having a slider assembly mounted therein. The slider assembly may include a slider having an internal fluid channel and a seal overlaying at least a portion of an outer surface of the slider, excluding the internal fluid channel, and a resilient member supporting the slider in the intermediate housing. When a user applies an axial force by pressing or otherwise pushing the slider, the force displaces or otherwise moves the slider downwards, thereby compressing the resilient member. As the axial force F continues to be applied, the slider may continue to be displaced from the initial position in the non-actuated state to a first position where the internal fluid channel of the slider may be axially aligned with internal fluid channels of the proximal, distal, and intermediate housings. Accordingly, fluid communication may be established between the proximal, distal, and intermediate housings and the slider to allow a fluid to flow from the distal housing to the proximal housing via the intermediate housing. The aligned internal fluid channels together may define a linear internal flow channel through the flow restriction device. As such, during blood collection or withdrawal from the patients, blood may flow from the patient's vein into a blood collection device via the internal flow channel of flow restriction device.

The flow restriction device of the various embodiments described herein is advantageous over currently existing blood collection systems. For example, during blood draw with currently existing blood draw devices, blood cells may experience shear stress as they flow from the distal housing to the proximal housing. The maximum shear stress may be along the wall of the blood cell, often referred to as wall shear stress. Wall shear stress on blood cells is considered a major source of mechanical damage to blood cells causing hemolysis of the blood cells. In some embodiments, the linear internal flow channel having minimal diameter may facilitate increased flow resistance within the vascular access system to distribute the pressure differential and reduce shear stress experienced by the red blood cells of the blood. For example, the minimized diameter of the internal flow channel may provide increased resistance to flow of the blood and thereby decrease blood flow rate within the flow resistance device. Since the decreased blood flow rate causes a reduction in shear stress experienced by the red blood cells of the blood, a risk of hemolysis during blood collection may advantageously be reduced.

When blood draw is completed and the user removes the axial force by releasing the slider, the resilient member pushes the slider back upwards towards the initial non-actuated (i.e., closed or second) position. The slider may move upwards, causing the internal fluid channel of the slider to not be aligned, or to be misaligned with the internal fluid channels of the proximal, distal, and intermediate housings. Accordingly, in the closed or second positon the seal, which overlays the outer surface of the slider, may block fluid connection between the proximal and distal housings.

The aforementioned configuration of the flow restriction device in which the internal fluid channel is not aligned with the internal fluid channels and the seal blocks fluid connection between the proximal and distal housings is advantageous over other blood collection systems in that it is capable of preventing blood spillage from the flow restriction device during disconnection from the blood collection device. For example, with other blood collection systems, the recommended blood draw procedures dictate that the blood draw connector should not be disconnected from blood collection device (e.g., luer lock access device (LLAD)) after completion of blood draw procedure. If the blood draw connector is disconnected from blood collection device, it may cause potential blood spillage from the blood draw connector channel when it is disconnected from blood collection device. In contrast, the flow restriction devices of the various embodiments described herein in which the internal fluid channel is not aligned with the internal fluid channels and the seal blocks fluid connection between the proximal and distal housings, allows for the blood to be contained within the internal fluid channel of the slider, thereby preventing blood spillage when the flow restriction device is disconnected from the blood collection device for disposal.

The flow restriction devices and associated blood collection systems of the various embodiments described herein additionally provide further advantages over currently existing blood collection systems. For example, add-on flow restriction devices described herein allow for hemolysis-reduction function to be integrated for PIVC blood draw. Further, the flow restriction devices described herein are compatible with PIVC placement and allow for seamless blood draw at insertion. Additionally, since the flow restriction devices are an add-on which can be easily incorporated without any changes to existing PIVC, there is minimal impact to clinical setting and operations.

FIG. 1 illustrates a vascular access device 100 including a peripheral intravenous catheter (PIVC) assembly 50 that includes a flow restriction device 110, in accordance with some embodiments of the present disclosure. The flow restriction device 110 may be configured to reduce a likelihood of hemolysis during blood collection using the vascular access device 100. In some embodiments, the vascular access device 100 may include a catheter assembly 50. The catheter assembly 50 may include a catheter hub 52, which may include a distal end 54, a proximal end 56, and a lumen extending through the distal end and the proximal end. The catheter assembly 50 may further include a catheter 58, which may be secured within the catheter hub 52 and may extend distally from the distal end 54 of the catheter hub 52. In some embodiments, the catheter assembly 50 may be a peripheral intravenous catheter (PIVC).

In some embodiments, the catheter assembly 50 may include or correspond to any suitable catheter assembly 50. In some embodiments, the catheter assembly 50 may be integrated and include an extension tube 60, which may extend from and be integrated with a side port 59 of the catheter hub 52. A non-limiting example of an integrated catheter assembly is the BD NEXIVA™ Closed IV Catheter system, available from Becton Dickinson and Company. In some embodiments, a proximal end of the extension tube 60 may be coupled to an adapter, such as, for example, a Y-adapter 70. In some embodiments, the flow restriction device 110 may be fluidly coupled to the Y-adapter 70.

In some embodiments, the catheter assembly 50 may be non-integrated and may not include the extension tube 60. In these and other embodiments, the flow restriction device 110 may be configured to couple to the proximal end 56 of the catheter hub 52 or another suitable portion of the catheter assembly 50. In some embodiments, the flow restriction device 110 may be coupled directly to the catheter assembly 50, eliminating the extension tube 60 and providing a compact catheter system.

In accordance with some embodiments, the flow restriction device 110 may be configured to couple to a blood collection device 40. For example, the flow restriction device 110 may include a female luer connector (e.g., proximal housing 112), which may be coupled with a male luer connector of the blood collection device 40. In some embodiments, the blood collection device 140 may be a luer lock access device (LLAD).

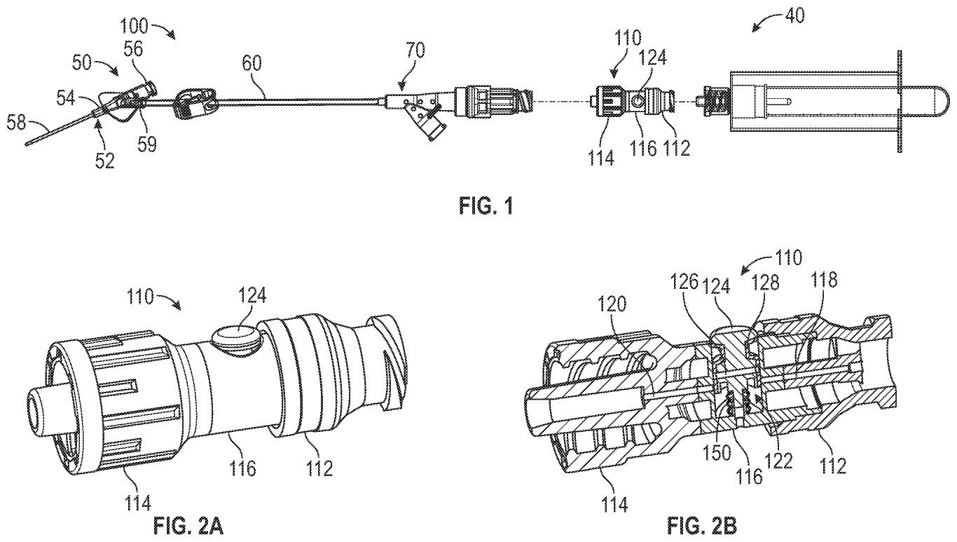

FIG. 2A illustrates a perspective view of the flow restriction device 110, in accordance with some embodiments of the present disclosure. FIG. 2B illustrates a cross-sectional view of the flow restriction device 110 of FIG. 2A, in accordance with some embodiments of the present disclosure. As depicted in FIGS. 2A and 2B, in some embodiments, the flow restriction device 110 may include a proximal housing 112 configured to couple to the blood collection device 40. For example, the proximal housing 112 may be integrated with the blood collection device 40 or monolithically formed with the blood collection device 40 as a single unit. As another example, the proximal housing 112 may include the female luer connector, which may be coupled with a male luer connector of the blood collection device 40.

In some embodiments, the flow restrictor device 110 may be in the form of a cylindrical body extending from the proximal housing 112 to the distal housing 116, with the intermediate housing interposed between the proximal and distal housings 112 and 114.

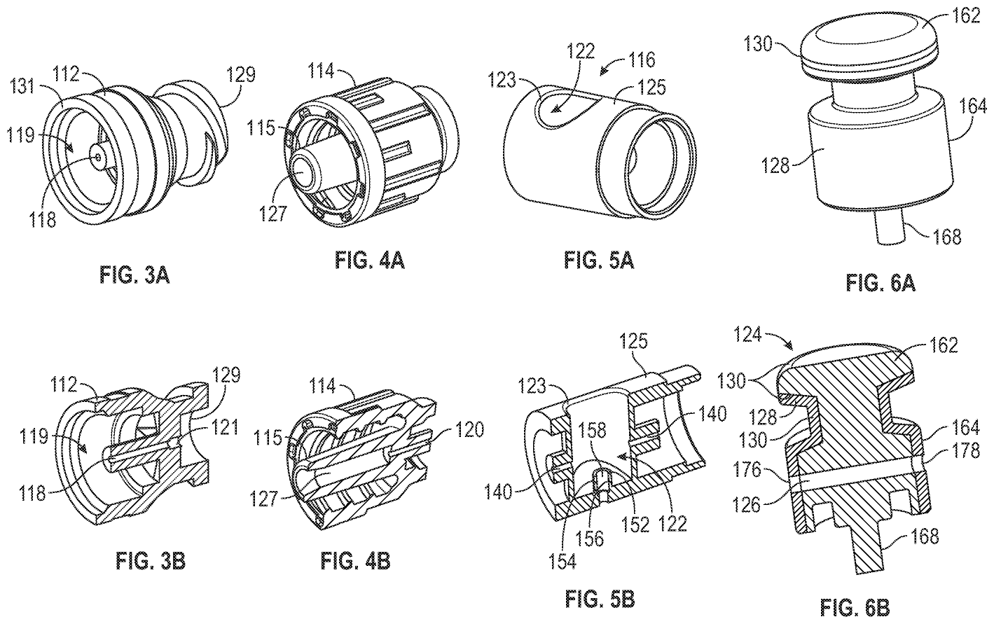

FIG. 3A illustrates a perspective view of a proximal housing of the flow restriction device, in accordance with some embodiments of the present disclosure. FIG. 3B illustrates a cross-sectional view of the proximal housing of FIG. 3A, in accordance with some embodiments of the present disclosure. In some embodiments, the proximal housing 112 may be in the form of a proximal connector or female luer connector having a first end 129 for coupling to a male luer portion of the blood collection device 40 and a second end 131 for coupling to the intermediate housing 116. The proximal housing 112 may include an internal fluid channel 118 extending transversely at least partially therethrough. As depicted in FIGS. 3A and 3B, with continued reference to FIG. 2B, the second end 131 may define a chamber 119 into which the intermediate housing 116 may be at least partially inserted. Accordingly, the internal fluid channel 118 may be coupled to and fluidly connected with an internal fluid channel 140 of the intermediate housing 116 at a first end of the intermediate housing 116.

FIG. 4A illustrates a perspective view of a distal housing of the flow restriction device, in accordance with some embodiments of the present disclosure. FIG. 4B illustrates a cross-sectional view of the distal housing of FIG. 4A, in accordance with some embodiments of the present disclosure. In some embodiments, the distal housing 114 may be in the form of a distal connector or male luer connector first end with a male luer portion 129 for fluidly coupling the distal housing 114 to the catheter assembly 50, and a second end 133 for coupling to the intermediate housing 116. Similar to the proximal housing 112, the distal housing 114 may include an internal fluid channel 120 extending transversely at least partially therethrough. As depicted in FIGS. 4A and 4B, with continued reference to FIG. 2B, the second end 133 be coupled to the internal fluid channel 140 of the intermediate housing 116 at a second end of the intermediate housing 116. Accordingly, the internal fluid channel 120 may be fluidly connected with the internal fluid channel 140 of the intermediate housing 116 at the second end of the intermediate housing 116.

FIG. 5A illustrates a perspective view of an intermediate housing of the flow restriction device, in accordance with some embodiments of the present disclosure. FIG. 5B illustrates a cross-sectional view of the intermediate housing of FIG. 5A, in accordance with some embodiments of the present disclosure. In some embodiments, the intermediate housing 116 may be in the form of a cylindrical body having a first end 116 coupled to the proximal housing 112, and a second end 117 coupled to the intermediate housing 116. Similar to the proximal and distal housings 112 and 114, the intermediate housing 116 may have an internal fluid channel 140 extending transversely therethrough on opposing sides of an internal chamber 122.

In some embodiments, the intermediate housing 116 may include an internal chamber 122 and a slider 124 reciprocally disposed in the internal chamber 122. The chamber 122 may extend through an outer surface of the intermediate housing 116 to an exterior thereof. For example, in some embodiments, the intermediate housing may include an aperture 123 extending from the chamber though the outer surface 125 of the intermediate housing. The aperture 123 may fluidly connect the internal chamber 122 and the exterior of the intermediate housing 116. Accordingly, in a non-actuated position of the slider 124, at least a portion of the slider 124 may extend to the exterior of the intermediate housing 116 via the aperture 123. Accordingly, a user may easily access and actuate the slider 124 from the exterior of the intermediate housing 116.

According to various embodiments of the present disclosure, the slider 124 may also include an internal fluid channel 126 extending transversly therethrough. As shall be described below, the internal fluid channels 118, 120, 126, and 140 may each be linear fluid channels which together define a linear internal flow channel 170 through the flow restriction device 110.

In some embodiments, the slider 124 may further include a seal 128 overlaying at least a portion of an outer surface 130 of slider 124. As depicted in FIG. 6B, the seal 128 may overlay the outer surface 130 without covering the internal fluid channel 126 of slider 124. For example, as depicted the seal 128 may not be overlaid over an inlet port 176 and an outlet port 178 of the internal fluid channel 126. Accordingly, flow into the internal fluid channel 126 may not be blocked or otherwise obstructed by the seal 128. In some embodiments, the seal may be a rubber seal. However, the various embodiments of the present disclosure are not limited to the aforementioned configuration, and the seal may be formed of any other material suitable for sealing and preventing leakages.

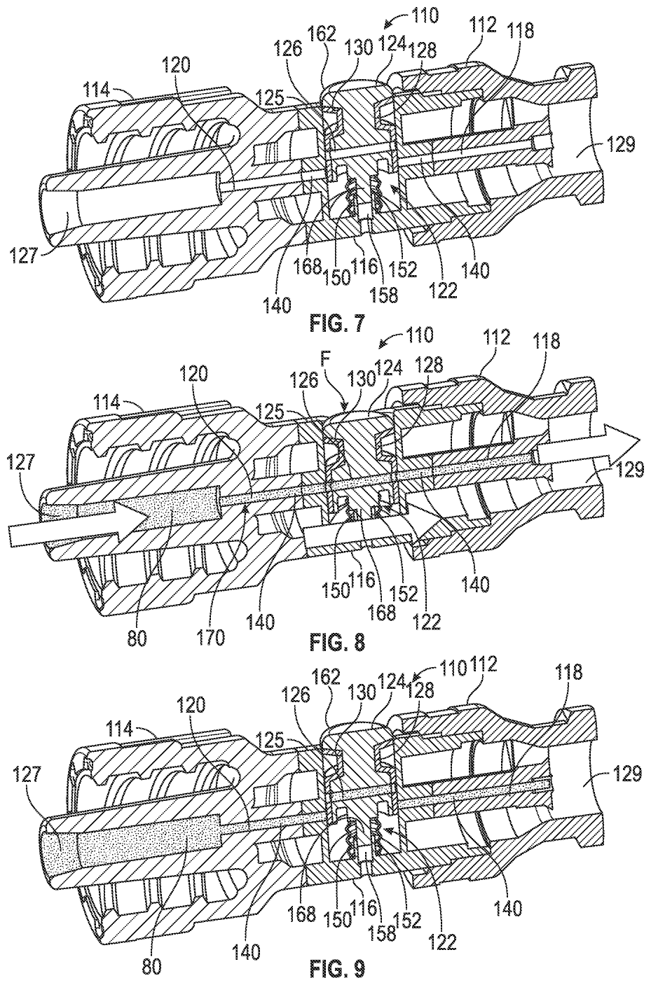

As shall be described in further detail below with respect to FIGS. 7-9, the slider 124 may be reciprocally movable between a first position (illustrated in FIG. 8) and a second position (illustrated in FIG. 9). In the first position, the internal fluid channel 126 of the slider 124 may be axially aligned with the internal fluid channels 118, 120, and 140 of the proximal, distal, and intermediate housings 112, 114, and 116 to allow a fluid to flow from the distal housing 114 to the proximal housing 112 via the intermediate housing 116. In the second position, the internal fluid channel 126 of the slider 124 is not aligned with the internal fluid channels 118, 120, and 140 of the proximal, distal, and intermediate housings 112, 114, and 116, and the seal 128 blocks fluid connection between the proximal and distal housings 112 and 114.

As further depicted, in some embodiments the intermediate housing 116 may further include a mount 152 disposed in the chamber 122 into which the slider 124 may be reciprocally mounted. As depicted, the mount 152 may have an outer surface 154 and an inner surface 156 defining a lumen 158 extending at least partially therethrough. At least a portion of the slider 124 may be reciprocally disposed and supported in the lumen 158 of the mount 152. For example, in some embodiments, the intermediate housing 116 may further include a resilient member 150 mounted in the chamber 122 and coupled to the slider 124 to support the slider within the lumen 158 of mount 152. As depicted, the resilient member 150 may be disposed around and coupled to the outer surface 154 of the mount 152 to support the slider 124 within the lumen 158. In some embodiments, the resilient member 150 may be a silicon spring, silicon bellows, a rubber spring, or rubber bellows.

According to various embodiments of the present disclosure the slider may include a head 162, a stem 168, and a body 164 disposed between the head and stem portions 162 and 168. In a normally closed position (illustrated in FIG. 7) and in the second position (illustrated in FIG. 9), the head 162 of the slider 124 may extend to the exterior of the intermediate housing 116. The aforementioned configuration is advantageous in that a user may easily access the slider 124 from the exterior of the flow restriction device 110.

According to various embodiments of the present disclosure, a method of manufacturing a flow restriction device may include molding the proximal housing 112 with the internal fluid channel 118 extending transversely at least partially therethrough, and molding the distal housing 114 with the internal fluid channel 120 extending transversely at least partially therethrough, and molding the intermediate housing 116 with the internal chamber 122 extending through the outer surface of the intermediate housing. In some embodiments, molding the intermediate housing may further include molding the intermediate housing 116 with the internal fluid channel 140 extending transversely at least partially therethrough at opposing ends of the chamber 122. Molding the intermediate housing 116 may further include molding the intermediate housing 116 with the mount 152 extending radially inward from the chamber 122 and including outer surface 154 and inner surface 156 defining the lumen 158 extending at least partially therethrough.

The method may further include molding the slider 124 with the internal fluid channel 126 extending transversly therethrough, and overmolding the seal 128 over at least a portion of the slider 124 excluding the internal fluid channel 126.

The method may further include mounting the slider 124 with overmolded seal 128 in the intermediate housing 116, and coupling the intermediate housing 116 between the proximal and distal housings 112 and 114. In some embodiments, mounting the slider with overmolded seal in the intermediate housing may further include coupling the resilient member 150 to the slider 124, and mounting the slider 124 at least partially in the lumen 158 with the resilient member 150 surrounding and further coupled to the outer surface 154 of the mount 152. In some embodiments, coupling the intermediate housing 116 between the proximal and distal housings 112 and 114 may include ultrasonically welding the intermediate housing 116 to the proximal and distal housings 112 and 114.

The operation of the flow restriction device 110 is described below with reference to FIGS. 7-9. FIG. 7 illustrates a cross-sectional view of the flow restriction device 110 in a non-actuated state of the slider 124, in accordance with some embodiments of the present disclosure. As illustrated in FIG. 7, in the absence of an axial force being applied to the slider 124, the resilient member 150 maintains the slider 124 in a where at least a portion of the slider is disposed exterior to the intermediate housing. For example, as illustrated in FIG. 7, absent the axial force F being applied, the slider 124 is disposed in the intermediate housing 116 with at least a portion of the head 162 disposed exterior to the intermediate housing 116. In the non-actuated state, the resilient member 150 biases the slider to the upper-most position where the internal fluid channel 126 is not aligned with the internal fluid channels 118, 120, and 140 of the proximal, distal, and intermediate housings 112, 114, and 116. In order for blood draw to occur and blood to flow from the catheter assembly 150 into the blood draw device 40, a user may actuate the slider 124 by pressing or otherwise pushing downwards on the slider 124.

FIG. 8 illustrates a cross-sectional view of the flow restriction device 110 in an actuated state of the slider 124, in accordance with some embodiments of the present disclosure. When the user applies an axial force F by pressing or otherwise pushing the slider 124 (e.g., the head 162 of the slider 124), the force F displaces or otherwise moves the slider 124 downwards towards the mount 152, thereby compressing the resilient member 150. As the axial force F continues to be applied, the slider 124 may continue to be displaced from the initial position in the non-actuated state to a first position. As depicted, in the first position, the internal fluid channel 126 of the slider 124 may be axially aligned with the internal fluid channels 118, 120, and 140 of the proximal, distal, and intermediate housings 112, 114, and 116. Accordingly, fluid communication may be established between the proximal, distal, and intermediate housings 112, 114, and 116, and the slider 124 to allow a fluid to flow from the distal housing 114 to the proximal housing 112 via the intermediate housing 116. The aligned internal fluid channels 118, 120, 126, and 140 together may define a linear internal flow channel 170 through the flow restriction device 110. In some embodiments, the linear internal flow channel 170 may have a minimal diameter as compared with various other lumens or fluid pathways of the system 100. For example, in some embodiments, the linear internal flow channel 170 may have a diameter ranging from about 0.02 inches to 0.03 inches, in some instances ranging from about 0.022 inches to 0.028 inches, more typically from about 0.024 inches to 0.026 inches, and in some embodiments approximately 0.025 inches. Though recited in terms of certain ranges, it will be understood that all ranges from the lowest of the lower limits to the highest of the upper limits are included, including all intermediate ranges or specific angles, within this full range or any specifically recited range.

In some embodiments, the linear internal flow channel 170 may have a length ranging from about 0.5 inches to 3 inches, in some instances ranging from about 0.7 inches to 2 inches, more typically from about 0.9 inches to 1.5 inches, and in some embodiments approximately 1 inch. Though recited in terms of certain ranges, it will be understood that all ranges from the lowest of the lower limits to the highest of the upper limits are included, including all intermediate ranges or specific angles, within this full range or any specifically recited range.

As such, during blood collection or withdrawal from the patients, the blood 80 may flow from the patient's vein into the catheter assembly 50, through the extension tubing 60, enter the internal flow channel 170 of flow restriction device 110 via the inlet flowpath 127 of the proximal housing 114, exit the flow restriction device 110 via the outlet flowpath 121 of the proximal housing 112, and enter the blood collection device 40. Accordingly, during blood collection or withdrawal from the patients, the blood 80 may flow into the blood collection device 40 via the internal flow channel 170 having minimal diameter. The flow restriction device 110 of the various embodiments described herein is advantageous over currently existing blood collection systems. For example, during blood draw with currently existing blood draw devices, blood cells may experience shear stress as they flow from the distal housing 114 to the proximal housing 112 of the flow restriction device 110. The maximum shear stress may be along the wall of the blood cell, often referred to as wall shear stress. Wall shear stress on blood cells is considered a major source of mechanical damage to blood cells causing hemolysis of the blood cells. In some embodiments, the linear internal flow channel 170 having minimal diameter may facilitate increased flow resistance within the vascular access system 100 to distribute the pressure differential and reduce shear stress experienced by the red blood cells of the blood 80. For example, the minimized diameter of the internal flow channel 170 may provide increased resistance to flow of the blood 80 and thereby decrease blood flow rate within the flow resistance device 110. Since the decreased blood flow rate causes a reduction in shear stress experienced by the red blood cells of the blood 80, a risk of hemolysis during blood collection may advantageously be reduced.

FIG. 9 illustrates a cross-sectional view of the flow restriction device 110 returned to the non-actuated state of the slider 124 after completion of fluid (e.g., blood) draw, in accordance with some embodiments of the present disclosure. When blood draw is completed and the user removes the axial force F by releasing the slider 124, the resilient member 150 pushes the slider 124 back upwards towards the initial non-actuated (i.e., closed or second) position. The slider 124 may move upwards, causing the internal fluid channel 126 of the slider 124 to not be aligned, or to be misaligned with the internal fluid channels 118, 120, and 140 of the proximal, distal, and intermediate housings 112, 114, and 116. Accordingly, in the closed or second positon the seal 128, which overlays the outer surface 130 of the slider 124, blocks fluid connection between the proximal and distal housings 112 and 114.

The aforementioned configuration of the flow restriction device 110 in which the internal fluid channel 126 is not aligned with the internal fluid channels 118, 120, and 140 and the seal 128 blocks fluid connection between the proximal and distal housings 112 and 114 is advantageous over other blood collection systems in that it is capable of preventing blood spillage from the flow restriction device 110 during disconnection from the blood collection device 40. For example, with other blood collection systems, the recommended blood draw procedures dictate that the blood draw connector should not be disconnected from blood collection device (e.g., luer lock access device (LLAD)) after completion of blood draw procedure. If the blood draw connector is disconnected from blood collection device, it may cause potential blood spillage from the blood draw connector channel when it is disconnected from blood collection device. In contrast, the flow restriction device 110 of the various embodiments described herein in which the internal fluid channel 126 is not aligned with the internal fluid channels 118, 120, and 140 and the seal 128 blocks fluid connection between the proximal and distal housings 112 and 114 allows for the blood to be contained within the internal fluid channel 126 of the slider 124, thereby preventing blood spillage when the flow restriction device 110 is disconnected from the blood collection device 40 for disposal.

The flow restriction devices and associated blood collection systems of the various embodiments described herein additionally provide further advantages over other blood collection systems. For example, add-on flow restriction devices described herein allow for hemolysis-reduction function to be integrated for PIVC blood draw and reduce hemolysis significantly. Further, the flow restriction devices described herein are compatible with PIVC placement and allow for seamless blood draw at insertion. Furthermore, since the flow restriction devices are an add-on which can be easily incorporated without any changes to existing PIVC, there is minimal impact to clinical setting and operations. Additionally, the flow restriction devices described herein minimize or otherwise eliminate the risk of blood spillage during disconnection from the blood draw device. Furthermore, the flow restriction devices described herein have low blood wastage due to a smaller priming volume as compared with other blood collection systems. In addition, the flow restriction devices described herein require less fill time as compared with currently existing blood collection systems.

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 5. The other clauses can be presented in a similar manner.

Clause 1. A flow restriction device, comprising: a proximal housing configured to couple to a fluid collection device and including an internal fluid channel extending transversely at least partially therethrough; a distal housing configured to couple to a catheter assembly and including an internal fluid channel extending transversely at least partially therethrough; and an intermediate housing interposed between the proximal and distal housings, the intermediate housing comprising: an internal chamber; and a slider reciprocally disposed in the internal chamber, the slider comprising an internal fluid channel extending transversely therethrough and a seal overlaying an outer surface of slider excluding the internal fluid channel, wherein the slider is reciprocally movable between (i) a first position where the internal fluid channel of the slider is axially aligned with the internal fluid channels of the proximal and distal housings to allow a fluid to flow from the distal housing to the proximal housing via the intermediate housing, and (ii) a second position where the internal fluid channel of the slider is not aligned with the internal fluid channels of the proximal and distal housings and the seal blocks fluid connection between the proximal and distal housings.

Clause 2. The flow restriction device of Clause 1, wherein the intermediate housing further comprises an internal fluid channel extending transversely therethrough on opposing sides of the internal chamber.

Clause 3. The flow restriction device of Clause 2, wherein the intermediate housing further comprises a resilient member mounted in the internal chamber and coupled to the slider, and in a normally closed state, the uncompressed resilient member biases the slider to the second position.

Clause 4. The flow restriction device of Clause 3, wherein when subject to an axial force, the resilient member compresses to move the slider from the normally closed state in the second position to the first position to allow flow through the internal fluid channels of the proximal, distal, and intermediate housings.

Clause 5. The flow restriction device of Clause 3, wherein the intermediate housing further comprises a mount disposed in the internal chamber and having an outer surface and an inner surface defining a lumen extending at least partially therethrough, at least a portion of the slider is reciprocally disposed in the lumen, and the resilient member is disposed around and coupled to the outer surface of the mount.

Clause 6. The flow restriction device of Clause 5, wherein the internal chamber extends through an outer surface of the intermediate housing to an exterior thereof, and the slider comprises a head, a body, and a stem.

Clause 7. The flow restriction device of Clause 6, wherein in the second position at least a portion of the head extends to the exterior of the intermediate housing.

Clause 8. The flow restriction device of Clause 3, wherein the resilient member comprises at least one of a silicon spring, silicon bellows, a rubber spring, or rubber bellows.

Clause 9. The flow restriction device of Clause 2, wherein the internal fluid channels of the proximal, intermediate, and distal housings and the slider through which the fluid flows from the distal housing to the proximal housing and into the fluid collection device comprise linear fluid channels defining a linear internal flow channel though the flow restriction device in the first position.

Clause 10. The flow restriction device of Clause 1, wherein the seal comprises a rubber seal.

Clause 11. The flow restriction device of Clause 1, wherein the fluid flowing from the proximal housing to the distal housing and into the fluid collection device comprises blood, and the fluid collection device comprises a blood collection device.

Clause 12. The flow restriction device of Clause 11, wherein the blood collection device comprises a luer lock access device.

Clause 13. A method of manufacturing a flow restriction device, comprising: molding a proximal housing with an internal fluid channel extending transversely at least partially therethrough; molding a distal housing with an internal fluid channel extending transversely at least partially therethrough; and molding an intermediate housing with an internal chamber extending through an outer surface of the intermediate housing; molding a slider with an internal fluid channel extending transversely therethrough; overmolding a seal over at least a portion of the slider excluding the internal fluid channel; mounting the slider with overmolded seal in the intermediate housing; and coupling the intermediate housing between the proximal and distal housings.

Clause 14. The method of Clause 13, wherein molding the intermediate housing further comprises molding the intermediate housing with an internal fluid channel extending transversely at least partially therethrough at opposing ends of the internal chamber.

Clause 15. The method of Clause 13, wherein molding the intermediate housing further comprises molding the intermediate housing with a mount extending radially inward from the internal chamber and including an outer surface and an inner surface defining a lumen extending at least partially therethrough.

Clause 16. The method of Clause 15, wherein mounting the slider with overmolded seal in the intermediate housing comprises coupling a resilient member to the slider, and mounting the slider at least partially in the lumen with the resilient member surrounding and further coupled to the outer surface of the mount.

Clause 17. The method of Clause 13, wherein coupling the intermediate housing between the proximal and distal housings comprises ultrasonically welding the intermediate housing to the proximal and distal housings.

Clause 18. A blood collection system, comprising: a blood collection device; a catheter assembly; and a flow restriction device fluidly coupled to the blood collection device, the flow restriction device comprising: an intermediate housing comprising: an internal chamber and an internal fluid channel extending therethrough on opposing sides of the internal chamber; and a slider reciprocally mounted in the internal chamber, the slider comprising an internal fluid channel extending transversely therethrough and a seal overlaying an outer surface of slider excluding the internal fluid channel; a distal housing coupled to the catheter assembly and including an internal fluid channel extending at least partially therethrough; and a proximal housing coupled to the blood collection device and including an internal fluid channel extending at least partially therethrough, wherein, in a non-actuated state of the slider the internal fluid channel of the slider is not aligned with the internal fluid channels of the proximal, distal, and intermediate housings and the seal blocks fluid connection between the proximal and distal housings.

Clause 19. The blood collection system of Clause 18, wherein in an actuated state of the slider, the internal fluid channel of the slider is axially aligned with the internal fluid channels of the proximal, distal, and intermediate housings to allow blood to flow from the distal housing to the proximal housing via the internal fluid channels.

Clause 20. The blood collection system of Clause 19, wherein the internal chamber extends through an outer surface of the intermediate housing to an exterior thereof, and the intermediate housing further comprises a resilient member mounted in the internal chamber and coupled to the slider, wherein in the non-actuated state, the uncompressed resilient member biases the slider to at least partially extend to the exterior of the intermediate housing.

Clause 21. The blood collection system of Clause 20, wherein when subject to an axial force, the resilient member compresses to move the slider from the non-actuated state to the actuated state to allow the blood to flow through the internal flow channels of the proximal, distal, and intermediate housings.

Clause 22. The blood collection system of Clause 20 wherein the intermediate housing further comprises a mount disposed in the internal chamber and having an outer surface and an inner surface defining a lumen extending at least partially therethrough, at least a portion of the slider is reciprocally disposed in the lumen, and the resilient member is disposed around and coupled to the outer surface of the mount.

Clause 23. The blood collection system of Clause 20, wherein the resilient member comprises at least one of a silicon spring, silicon bellows, a rubber spring, or rubber bellows.

Clause 24. The blood collection system of Clause 18, wherein the seal comprises a rubber seal.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, or operations in the processes or methods disclosed are illustrations of exemplary approaches. Based upon implementation preferences or scenarios, it is understood that the specific order or hierarchy of steps, operations or processes may be rearranged. Some of the steps, operations or processes may be performed simultaneously. In some implementation preferences or scenarios, certain operations may or may not be performed. Some or all of the steps, operations, or processes may be performed automatically, without the intervention of a user. The accompanying method claims present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but are to be accorded the full scope consistent with the language of the claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claim is:

1. A flow restriction device, comprising:
a proximal housing configured to couple to a fluid collection device and including an internal fluid channel extending transversely at least partially therethrough;
a distal housing configured to couple to a catheter assembly and including an internal fluid channel extending transversely at least partially therethrough; and
an intermediate housing interposed between the proximal and distal housings, the intermediate housing comprising:
an internal chamber; and
a slider reciprocally disposed in the internal chamber, the slider comprising an internal fluid channel extending transversly therethrough and a seal overlaying an outer surface of slider excluding the internal fluid channel,
wherein the slider is reciprocally movable between (i) a first position where the internal fluid channel of the slider is axially aligned with the internal fluid channels of the proximal and distal housings to allow a fluid to flow from the distal housing to the proximal housing via the intermediate housing, and (ii) a second position where the internal fluid channel of the slider is not aligned with the internal fluid channels of the proximal and distal housings and the seal blocks fluid connection between the proximal and distal housings.

2. The flow restriction device of claim 1, wherein the intermediate housing further comprises an internal fluid channel extending transversely therethrough on opposing sides of the internal chamber.

3. The flow restriction device of claim 2, wherein the intermediate housing further comprises a resilient member mounted in the internal chamber and coupled to the slider, and in a normally closed state, the uncompressed resilient member biases the slider to the second position.

4. The flow restriction device of claim 3, wherein when subject to an axial force, the resilient member compresses to move the slider from the normally closed state in the second position to the first position to allow flow through the internal fluid channels of the proximal, distal, and intermediate housings.

5. The flow restriction device of claim 3, wherein the intermediate housing further comprises a mount disposed in the internal chamber and having an outer surface and an inner surface defining a lumen extending at least partially therethrough, at least a portion of the slider is reciprocally disposed in the lumen, and the resilient member is disposed around and coupled to the outer surface of the mount.

6. The flow restriction device of claim 5, wherein the internal chamber extends through an outer surface of the intermediate housing to an exterior thereof, and the slider comprises a head, a body, and a stem.

7. The flow restriction device of claim 6, wherein in the second position at least a portion of the head extends to the exterior of the intermediate housing.

8. The flow restriction device of claim 2, wherein the internal fluid channels of the proximal, intermediate, and distal housings and the slider through which the fluid flows from the distal housing to the proximal housing and into the fluid collection device comprise linear fluid channels defining a linear internal flow channel though the flow restriction device in the first position.

9. The flow restriction device of claim 1, wherein the fluid flowing from the proximal housing to the distal housing and into the fluid collection device comprises blood, and the fluid collection device comprises a blood collection device.

10. The flow restriction device of claim 9, wherein the blood collection device comprises a luer lock access device.

11. A method of manufacturing a flow restriction device, comprising:
molding a proximal housing with an internal fluid channel extending transversely at least partially therethrough;
molding a distal housing with an internal fluid channel extending transversely at least partially therethrough; and
molding an intermediate housing with an internal chamber extending through an outer surface of the intermediate housing;
molding a slider with an internal fluid channel extending transversly therethrough;
overmolding a seal over at least a portion of the slider excluding the internal fluid channel;
mounting the slider with overmolded seal in the intermediate housing; and
coupling the intermediate housing between the proximal and distal housings.

12. The method of claim 11, wherein molding the intermediate housing further comprises molding the intermediate housing with an internal fluid channel extending transversely at least partially therethrough at opposing ends of the internal chamber.

13. The method of claim 11, wherein molding the intermediate housing further comprises molding the intermediate housing with a mount extending radially inward from the internal chamber and including an outer surface and an inner surface defining a lumen extending at least partially therethrough.

14. The method of claim 13, wherein mounting the slider with overmolded seal in the intermediate housing comprises coupling a resilient member to the slider, and mounting the slider at least partially in the lumen with the resilient member surrounding and further coupled to the outer surface of the mount.

15. The method of claim 11, wherein coupling the intermediate housing between the proximal and distal housings comprises ultrasonically welding the intermediate housing to the proximal and distal housings.

16. A blood collection system, comprising:
a blood collection device;
a catheter assembly; and
a flow restriction device fluidly coupled to the blood collection device, the flow restriction device comprising:
an intermediate housing comprising:
an internal chamber and an internal fluid channel extending therethrough on opposing sides of the internal chamber; and
a slider reciprocally mounted in the internal chamber, the slider comprising an internal fluid channel extending transversly therethrough and a seal overlaying an outer surface of slider excluding the internal fluid channel;
a distal housing coupled to the catheter assembly and including an internal fluid channel extending at least partially therethrough; and
a proximal housing coupled to the blood collection device and including an internal fluid channel extending at least partially therethrough,
wherein, in a non-actuated state of the slider the internal fluid channel of the slider is not aligned with the internal fluid channels of the proximal, distal, and intermediate housings and the seal blocks fluid connection between the proximal and distal housings.

17. The blood collection system of claim 16, wherein in an actuated state of the slider, the internal fluid channel of the slider is axially aligned with the internal fluid channels of the proximal, distal, and intermediate housings to allow blood to flow from the distal housing to the proximal housing via the internal fluid channels.

18. The blood collection system of claim 17, wherein the internal chamber extends through an outer surface of the intermediate housing to an exterior thereof, and the intermediate housing further comprises a resilient member mounted in the internal chamber and coupled to the slider, wherein in the non-actuated state, the uncompressed resilient member biases the slider to at least partially extend to the exterior of the intermediate housing.

19. The blood collection system of claim 18, wherein when subject to an axial force, the resilient member compresses to move the slider from the non-actuated state to the actuated state to allow the blood to flow through the internal flow channels of the proximal, distal, and intermediate housings.

20. The blood collection system of claim 18, wherein the intermediate housing further comprises a mount disposed in the internal chamber and having an outer surface and an inner surface defining a lumen extending at least partially therethrough, at least a portion of the slider is reciprocally disposed in the lumen, and the resilient member is disposed around and coupled to the outer surface of the mount.
